(19) United States
(12) Patent Application Publication
(10) Pub. No.: US 2026/0182949 A1
(43) Pub. Date: Jul. 2, 2026

(54) SYSTEM AND METHOD TO DETERMINE PHYSIOLOGICAL PARAMETERS OF A PERSON WITH THE USE OF HEART SOUND WAVEFORMS RETRIEVED USING AN EARPIECE DEVICE

(71) Applicant: OHMIC TECHNOLOGIES INC., Montreal (CA)

(72) Inventors: Carlos Antonio MENDES DA COSTA JUNIOR, Brossard (CA); Lino Bernard Bianca VAN MULKEN, Eindhoven (NL)

(21) Appl. No.: 19/545,872

(22) Filed: Feb. 20, 2026

Related U.S. Application Data
(63) Continuation of application No. PCT/IB2024/058104, filed on Aug. 20, 2024.
(60) Provisional application No. 63/533,873, filed on Aug. 21, 2023.

(51) Int. Cl. A61B 7/04; A61B 5/00; A61B 5/0205; A61B 5/021; H04R 1/46; H04R 5/033; H04S 1/00

(52) U.S. Cl. CPC A61B 7/04; A61B 5/0205; A61B 5/681; A61B 5/7264; H04R 1/46; H04R 5/033; H04S 1/007; A61B 5/021

(57) ABSTRACT

A system and method for determining heart-related biometric data is presented that includes an earpiece device device to receive audio signals and isolate right and left heartbeat signals from the received audio signals, an extraction module to process the isolated right and left heartbeat signals to generate right and left PCG1, PCG2 signals, a pre-processing module to perform signal processing on the PCG1, PCG2 signals to determine PTT11, PTT12 signals, and a processing module to perform signal processing on the PTT11, PTT12 data signals to provide an indication of heart-related conditions. Furthermore, incorporating morphological detection techniques on the PCG1, PCG2, PTT11, PTT12 signals to generate an MD data signal and incorporating a user-wearable device to generate a third heart-related signal that is further processed to generate PTT2, PTT3 data signals.

### CROSS-REFERENCE TO RELATED APPLICATIONS

The present application is a continuation of PCT Application No. PCT/IB2024/058104, with an international filing date of Aug. 20, 2024, which claims priority to U.S. Provisional Patent Application No. 63/533,873 , filed Aug. 21, 2023 entitled “Method to Determine Physiological Parameters of a Person with the Use of Heart Sound Waveforms Retrieved Using Headphones,” which is incorporated by reference herein in its entirety.

### TECHNICAL FIELD

The present disclosure generally relates to measuring biometrics and, in particular, measuring blood pressure and heart conditions with the use of an earpiece device and an apparatus communicatively-coupled to the earpiece device.

### BACKGROUND

Globally, cardiovascular diseases (CVDs) are the leading cause of mortality with hypertension being the main risk factor [1]. A timely diagnosis and proper treatment can prevent a large number of these mortalities [2]. Blood pressure is an important metric to diagnose CVDs [3]. Several methods are used to assess the blood pressure, such as oscillometry, ultrasound, volume clamping, and catheterization [4], where invasive methods are the most direct and accurate. However, such methods are technically demanding and unsuitable for assessing large populations [5]. Therefore, for routine assessments, methods incorporating cuff-based measurement techniques are used. A disadvantage of cuff-based measurements is that it is non-continuous, usually bulky, and can be discomforting for sensitive patients.

As a result, recently multiple cuffless methods have been explored, that enable patients to continuously measure blood pressure remotely while minimally affecting a patient's ambulatory or daily movement routines. Cuffless-based measurement methods frequently use Pulse Wave Velocity (PWV) signal, which is calculated from the Pulse Arrival Time (PAT) or Pulse Travel Time (PTT) signals over a fixed distance, as a metric, due to it being directly related to the blood pressure [3]. In most methods, the PAT signal is detected using Electrocardiography (ECG) and/or Photoplethysmography (PPG) measurements taken from a distal location from the heart on a patient's body.

Another method used to detect PAT/PTT signals are Phonocardiogram (PCG) techniques that use high-fidelity audio waveforms to estimate blood pressure and identify and/or diagnose heart-related issues including murmurs. However, PCG techniques are rarely used for remote/portability PAT/PTT signal monitoring, as PCG waveforms are typically measured locally by a stethoscope, thus making it difficult to keep the measurement device portable and easy to use. Recently, microphones have been proposed to detect PCG waveforms which, while not limiting portability, typically require extra sensors and hardware, thereby increasing the cost and size of the measurement device placed on a patient's body.

With this said, it will be appreciated that earpiece devices, such as headsets, headphones earbuds, and other similar devices, which have been a popular audio accessory for many years, have recently technologically advanced, to not only provide a high fidelity immersive stereo experience, but also incorporate a variety of additional features and functionalities, such as, noise cancelling technologies to minimize external ambient sounds/noises. Such advancements in earpiece technology have turned such devices into versatile multi-purpose devices that have the technological ability to also incorporate health monitoring functionalities.

Likewise, given the typically older demographics of hearing aid users, this technology could be extended to include hearing aids devices to estimate blood pressure and diagnose heart-related issues, such as murmurs.

### SUMMARY

The embodiments of the present disclosure have been designed based on the developers'appreciation of the drawbacks and issues associated with current apparatuses and methods.

In accordance with the disclosed embodiments, there is provided system for determining heart-related biometric data, that includes an earpiece device configured to receive audio signals and isolate right and left heartbeat signals from the received audio signals; an extraction module communicatively-coupled to the earpiece device and configured to process the isolated right and left heartbeat signals to generate respective right and left phonocardiogram PCG1, PCG2 data signals; and a processing host comprising a pre-processing module and a processing module. The pre-processing module is configured to perform signal processing measures on the right and left PCG1, PCG2 signals to determine respective pulse travel time PTT11, PTT12 data signals, and the processing module is configured to perform signal processing measures on the pulse travel time PTT11, PTT12 data signals to provide an indication of heart-related conditions.

In some aspects, the earpiece device comprises headsets, headphones, hearing aids device, or earbud set that is configured to provide independent right and left audio signals and isolate right and left heartbeat signals from the received audio signals.

In some aspects, the pre-processing module is further configured to perform signal processing measures that include morphological detection (MD) techniques on the PCG1, PCG2, PTT11, PTT12 data signals to generate an MD data signal indicative of heart murmur and/or related heart valve issues as well as generates diastolic period DP1, DP2 data signals and systolic period SP1, SP2 data signals, based on the PCG1, PCG2, PTT11, PTT12 data signals.

In some aspects, the system further comprises a user-wearable device including one or more sensors configured to detect and generate a third heart-related signal for processing by pre-processing module and processing module.

In some aspects, the signal processing measures of the processing module incorporate artificial intelligence (AI) deep-learning generated algorithms to the PTT11, PTT12 data signals, and/or MD, DP1, DP2, SP1, SP2 data signals to provide an indication of heart-related conditions.

In accordance with the disclosed embodiments, there is also provided a method for for determining heart-related biometric data that includes receiving, from an earpiece device, audio signals; isolating right and left heartbeat signals from the received audio signals; generating right and left phonocardiogram signals PCG1, PCG2 from the isolated right and left heartbeat signals, respectively; pre-processing, by a pre-processing module, the right and left PCG1, PCG2 signals to determine respective pulse travel time signals PTT11, PTT12 data signals; and processing, by a processing module, the PTT11, PTT12 data signals to provide an indication of heart-related conditions.

In some aspects, the method further comprises applying morphological detection (MD) to the PCG1, PCG2, PTT11, PTT12 data signals to generate an MD data signal indicative of heart murmurs as well as generating diastolic period DP1, DP2 data signals and systolic period SP1, SP2 data signals based on the PCG1, PCG2, PTT11, PTT12 data signals.

In some aspects, the method further comprises applying artificial intelligence (AI) deep-learning generated algorithms to the PTT11, PTT12 data signals, and/or MD, DP1, DP2, SP1, SP2 data signals to provide an indication of heart-related conditions.

In some aspects, the method further comprising generating, by a user-wearable device containing sensor(s), a third heart-related signal; and performing pre-processing and processing of the third heart-related signal.

The present technology provides a system for determining blood pressure and other heart-related biometrics to identify and/or diagnose heart conditions. To achieve these metrics, an apparatus is used to acquire two PCG signals from a headset (headphones, earbuds, hearing aids, or similar device worn by a person). The PCG signals are processed, where among other parameters, at least two PTTs are calculated. In addition to this, Morphological Detection (MD) is applied to the signals to determine the duration of the systolic and diastolic period and indicate any murmurs that have occurred. These metrics are then used in processing techniques and AI models to determine the blood pressure and other heart-related biometrics to diagnose heart conditions.

Furthermore, the present technology can be expanded by adding a third sensor, for example to measure an ECG, PCG or PPG signal. Thus, making it possible to calculate among other parameters, two or more additional PTTs, which are, likewise, used in AI models to determine the blood pressure, other biometrics and to diagnose heart conditions.

It will be appreciated that additional and/or alternative features, aspects, and advantages of the present technology will become apparent from the following description, accompanying drawings, and the appended claims.

### BRIEF DESCRIPTION OF THE DRAWINGS

For a more complete understanding of the present technology, reference is made to the following description and accompanying drawings, in which:

FIG. 1A depicts a monitoring arrangement for determining blood pressure and related biometrics that includes the use of ear-piece devices (headphones), in accordance with the embodiments of the present disclosure;

FIG. 1B depicts a schematic representation of an ear-piece devices (headphones) configured to determine blood pressure and related biometrics, in accordance with the embodiments of the present disclosure;

FIG. 1C depicts a high-level functional block diagram of a system incorporating ear-piece devices (headphones) for determining blood pressure and related biometrics, in accordance with the embodiments of the present disclosure;

FIG. 2A depicts an additional monitoring arrangement for determining blood pressure and related biometrics that includes the use of ear-piece devices (headphones) and wearable devices, in accordance with the embodiments of the present disclosure;

FIG. 2B depicts a high-level functional block diagram of an apparatus that includes the ear-piece devices (headphones) and wearable devices for determining blood pressure and related biometrics, in accordance with the embodiments of the present disclosure;

FIG. 3A depicts a flowchart of a method for determining blood pressure and heart-related biometric data based on inputs from a headset, in accordance with the embodiments of the present disclosure; and

FIG. 3B depicts a flowchart of a method for determining blood pressure and heart-related biometric data based on inputs from a headset and a wearable device, in accordance with the embodiments of the present disclosure.

It is to be understood that throughout the appended drawings and corresponding descriptions, like features are identified by like reference characters and that the drawings are not to scale. It should also be understood that the drawings and ensuing descriptions are intended for illustrative purposes only and that such disclosures are not intended to limit the scope of the claims.

### DETAILED DESCRIPTION OF THE EMBODIMENTS

The present disclosure introduces a new method to determine heart-related biometrics, such as, but not limited to, blood pressure to potentially diagnose heart conditions, with the use of an apparatus that generates PCG signals from diaphragm-pressure sensing excitation energy received from ear-piece devices.

It will be understood, however, that the examples and conditional language recited herein are principally intended to aid the reader in understanding the principles of the present technology and not to limit its scope to such specifically recited examples and conditions. It will be appreciated that those skilled in the art may devise various arrangements that, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity. In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology.

Moreover, where no examples of modifications have been set forth, it should not be interpreted that no modifications are possible and/or that what is described is the sole manner of implementing that element of the present technology. As such, all statements herein reciting principles, aspects, and implementations of the present technology, as well as specific examples thereof, are intended to encompass both structural and functional equivalents thereof, whether they are currently known or developed in the future.

It will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes that may be substantially represented in non-transitory computer-readable media and so executed by a computer or processor, whether or not such computer or processor is explicitly shown.

Similarly, functions of the various elements shown in the figures, including any functional block labeled as a “processor”, may be provided through the use of dedicated hardware as well as hardware capable of executing software in association with appropriate software.

Additionally, to the extent that the phrase “at least one of A and B” is used in the description and claims, it will be understood that this phrase is intended to mean “A only”, “B only” or both “A and B”.

With these fundamentals in place, presented heretofore are non-limiting embodiments that illustrate various aspects and implementations of the present disclosure.

The present technology is directed to a monitoring system and method for determining blood pressure and other heart-related biometrics to identify and/or diagnose heart conditions. The disclosed techniques employ certain components described in FAN [6] and certain headphone configurations described in co-pending U.S. Provisional Patent Application No. 63/524,528, filed Jun. 30, 2023 and PCT Application No. PCT/IB2024/056374, filed on Jun. 29, 2024 both entitled “Methods for Signal Extraction Using Stereo Audio Devices” to MENDES, Carlos et al. (hereinafter “MENDES”), which are incorporated by reference herein in their entireties.

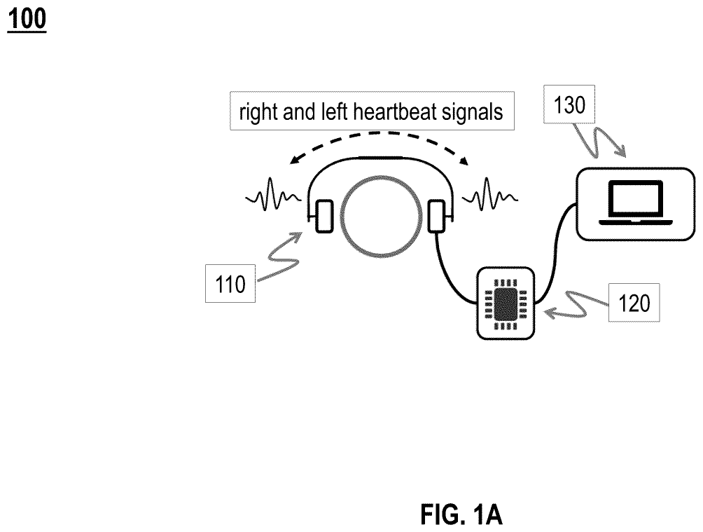

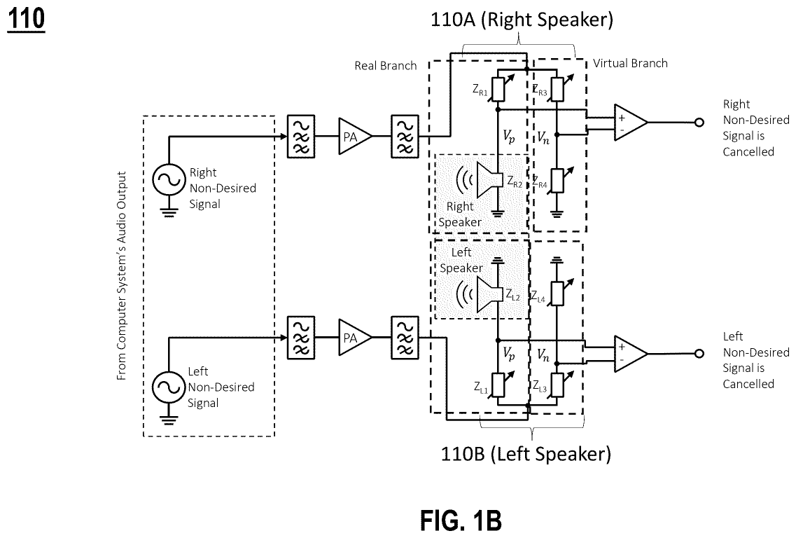

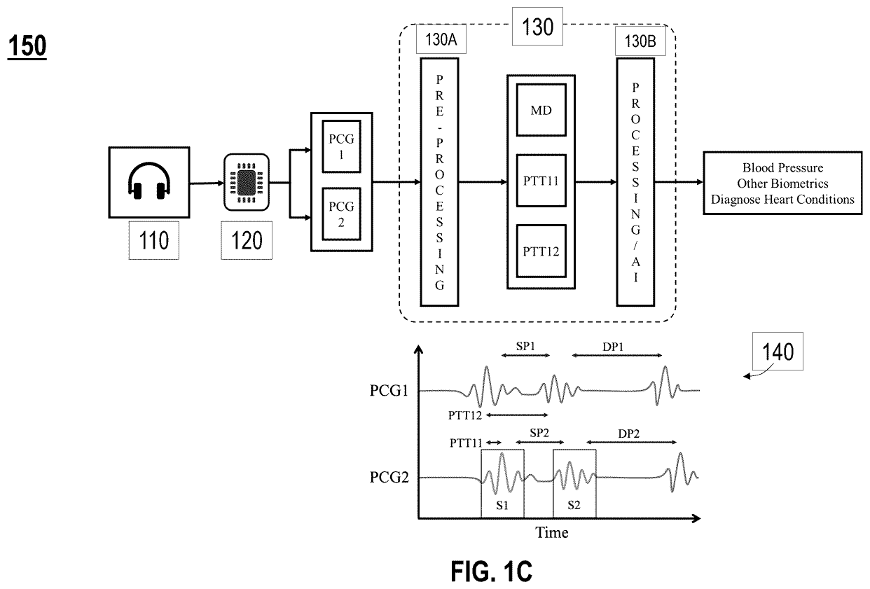

FIG. 1A depicts a monitoring arrangement 100 for determining blood pressure and heart-related biometric data that employs an earpiece device 110, in accordance with the embodiments of the present disclosure. For the sake of consistency and tractability, the ear-piece device 110 is shown to be in the form of headphones. However, it will be appreciated that headset 100 may also, without limitation, embody any user-wearable device having speaker functionality, such as headsets, earbuds, hearing aids, etc.

As shown, headphones 110 are communicatively-coupled to extraction module 120 that is configured to generate individually received left and right ear Phonocardiogram (PCG) signals PCG1, PCG2 representing heart sound recordings which can then be processed to generate heart-related biometrics and/or heart condition diagnoses.

By employing headphones 110 there is no need for microphones because, as noted above, microphones typically require extra sensors/hardware to record/measure the PCG signals.

FIG. 1B depicts a schematic representation of the configuration of the stereophonic headphones 110 designed to determine blood pressure and heart-related biometrics, in accordance with the embodiments of the present disclosure.

As shown, the stereophonic headphones 110 comprise a right speaker channel 110A and a left speaker channel 110B that play stereo audio signals while also being able to detect heart beat signals. By way of clarity, in this context, the stereo audio signals are referred to as “right and left undesired audio signals” while the detected heartbeat signals are referred to as “right and left desired monitoring signals.” It will be appreciated that, by detecting the desired heartbeat monitoring signal received by the right ear as well as receiving the desired heartbeat monitoring signal by the left ear, the detected heart beats are received from different location points on the body.

With this said, for blood pressure monitoring/determination processing, the headphones 110 are configured to cancel the right and left undesired audio signals to isolate the desired detected right and left heartbeat signals without interrupting the patient's listening of the right and left undesired audio signals.

Turning back to FIG. 1B, right and left speakers 110A, 110B respectively receive the right and left undesired audio signals after some filtering and amplification. As shown, each of the right and left speakers 110A, 110B comprise a real branch in parallel with a virtual branch, consistent with a Maxwell-Wien Bridge configuration. That is, the right speaker real branch includes variable impedance Z_{R1} and right speaker impedance Z_{R2} while the right speaker virtual branch includes variable impedances Z_{R3}, Z_{R4}. During blood pressure monitoring/determination processing, the right speaker virtual branch variable impedances Z_{R3}, Z_{R4} are adjusted to match the right speaker real branch impedances. As such, the right speaker real branch correspondingly generates a voltage signal Vₚ containing both the desired detected heartbeat signals and the undesired right audio signals while the right speaker virtual branch correspondingly generates a voltage signal Vₙ containing only the undesired right audio signals.

Similarly, the left speaker real branch includes variable impedance Z_{L1} and left speaker impedance Z_{L2} while the left speaker virtual branch includes variable impedances Z_{L3}, Z_{L4}. During blood pressure monitoring/determination processing, the left speaker virtual branch variable impedances Z_{L3}, Z_{L4} are adjusted to match the right speaker real branch impedances. Then left speaker real branch correspondingly generates a voltage signal Vₚ containing both the desired detected heart beat signals and the undesired left audio signals while the left speaker virtual branch correspondingly generates a voltage signal Vₙ containing only the undesired left audio signals.

Then, as shown, each of the right and left speakers 110A, 110B respectively incorporate subtraction elements that function to perform Vₚ-Vₙ operations, such that the right and left undesired audio signals are cancelled to yield only the right and left desired detected heartbeat signals for blood pressure monitoring/determination processing. Again, the cancellation of the right and left undesired audio signals does not, in any way, disrupt the patient's listening of the right and left undesired audio signals.

Armed with the noted configuration of headphones 110, FIG. 1C depicts a high-level functional block diagram of system 150 for determining blood pressure and heart-related biometric data and/or heart conditions, in accordance with the embodiments of the present disclosure. As shown, system 150 comprises the stereophonic headphones 110 that isolate the right and left heartbeat signals, PCG extraction module 120 that generates the right and left PCG signals from the isolated heartbeat signals, and processing host device 130 that generates the blood pressure, heart-related biometric data, and/or heart condition diagnoses.

In particular, the stereophonic headphones 110 supply the isolated right and left heartbeat signals to the PCG extraction module 120, which processes the isolated heartbeat signals to extract and generate the right and left PCG1, PCG2 waveforms. As noted above, PCG records heart sounds during a cardiac cycle. Because in stereo implementations, the right and left signals can be extracted independently, in some embodiments, the right signal may be grounded while allowing the left signal to be processed and vice versa generate the right and left PCG1, PCG2 signals. The grounding may be performed through software or hardware control of the PCG extraction module 120. In other embodiments, instrumentation or differential amplifiers may be employed to determine the differences between the right and left signals to generate the right and left PCG1, PCG2 signals. In yet other embodiments, both signals can be captured at the same time by a dual channel Analog-to-Digital Converter (ADC), instead of being captured non-simultaneously by grounding one or the other. Then, the generated right and left PCG1, PCG2 signals are then supplied to processing host 130 which, as noted above, may comprise a computer, smartphone, smart watch, etc.

As shown in FIG. 1C, the processing host 130 comprises a pre-processing module 130A and a processing module 130B. The pre-processing module 130A is configured to perform various signal processing steps on the received independent right and left PCG1, PCG2 signals, such as, filtering, amplification, noise mitigation, etc. to determine the two Pulse Travel Time (PTT) signals, namely PTT11, PTT12, in accordance with the distance between the two different body location points, as collected by the right ear PCG signal and the left ear PCG signal. In particular, PCG1 is captured from one ear (e.g., the right ear) and PCG2 is captured from the other ear (e.g., the right ear) of the patient. The first and second peak of each of the PCG1, PCG2 signals are referred to as S1 and S2 respectively. The time delay between the arrival time of S1 of PCG1 and PCG2 are referred to as PTT11. Likewise, the time-delay between the S1 of PCG1 and S2 of PCG2 are referred to as PTT12.

Moreover, the pre-processing module 130A may additionally incorporate morphological detection (MD) techniques, based on the right and left PCG1, PCG2 signals and the determined PTT11, PTT12, to generate an MD signal indicative of any heart murmurs and/or heart valve issues.

The PTT11, PTT12, and MD signal determinations are then supplied to processing module 130B. In some embodiments, additional biometric data, such as, diastolic DP1, DP2 period data, systolic SP1, SP2 period data, and PCG1, PCG2 signals may also be supplied to the processing module 130B.

Processing module 130B is configured to provide additional signal processing steps, such as, filtering, amplification, noise mitigation, timing correlations, etc. as well as applying AI deep learning generated algorithms to identify the heart-related biometric data that may indicate any heart-related conditions, such as, blood pressure measurements along with heart murmur, heart valve, and blood vessel issue detection. Accordingly, FIG. 1C illustrates an exemplary graph 140 representing the heart-related data, in waveform fashion, generated by processing module 130B.

As shown, graph 140 depicts data representing the PCG1, PCG2, PTT11, and PTT12 signals that are used to evaluate potential heart-related conditions. In particular, graph 140 indicates two heart beats S1 and S2 as well as the systolic period data SP1, SP2 and diastolic period data DP1, DP2 due to the periodic contractions and expansions of the heart during a cardiac cycle. The first and second peak of each PCG signal is referred to as S1, S2, respectively. The time-delay between the arrival time of S1 of PCG1 and PCG2 is referred to as PTT11, while the time-delay between the S1 of PCG1 and S2 of PCG2 is referred to as PTT12. The time between the S1, S2 and S2, S1 are referred to as the systolic period (SP) and diastolic period (DP) respectively.

The time between the S1 and S2, S2 and S1 is called the Systolic Period (SP) and Diastolic Period (DP) respectively. Therefore, the metrics calculated from the retrieved PCG signals include, but not limited to, PTT11, PTT12, SP1, SP2, DP1 and DP2.

In this manner, system 150 provides the heart-related biometric data, namely, in the form of PTT11, PTT12, SP1, SP2, DP1 and DP2 signal data to enable the diagnoses of heart-related conditions without subjecting patients to intrusive or movement-restrictive procedures. Such heart-related conditions may include, but are not limited to, blood pressure measurements along with heart murmur, heart valve, and blood vessel issue detection.

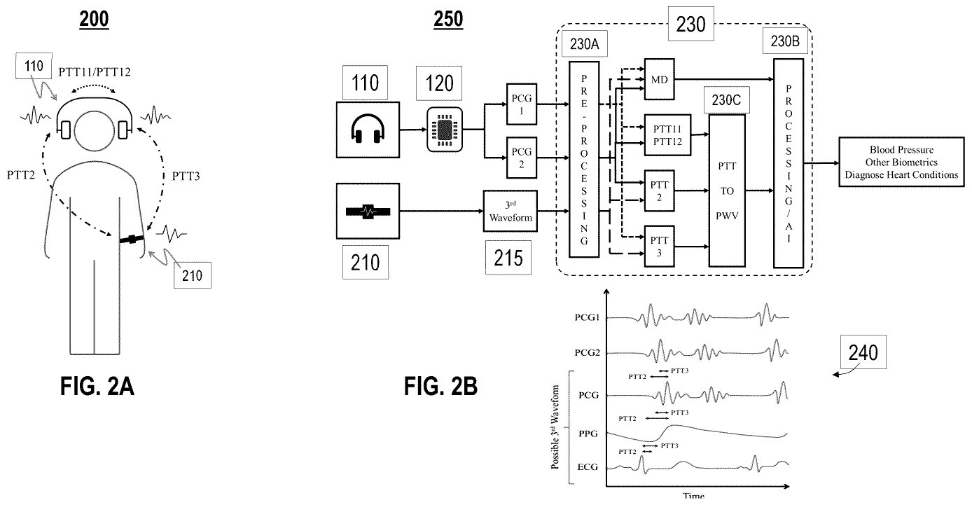

FIG. 2A depicts an additional monitoring arrangement 200 for determining blood pressure and related biometrics that includes the use of an earpiece device and wearable device, in accordance with the embodiments of the present disclosure. As shown, arrangement 200 includes an earpiece device 110 and a user-wearable device 210 located at a distal body position away from headset 110. As noted above, for the sake of consistency and tractability, the earpiece device 110 is shown to be in the form of headphones, but may also comprise headsets, earbuds, hearing aids, etc. Moreover, user-wearable device 210 may comprise a smartwatch, smart wrist/ankle/arm/leg/finger band/ring or any wearable device configured with sensor(s) capable of detecting heart-related signals. Much like arrangement 100 detailed above, the headphones 110 of arrangement 200 provide the raw isolated right and left heartbeat signals for processing. However, arrangement 200 provides for an additional heart-related signal data detected by user-wearable device 210 sensor(s).

Along these lines, FIG. 2B depicts a high-level functional block diagram of system 250 that utilizes headphones 110 and user-wearable device 210 for detecting heart-related biometric data, in accordance with the embodiments of the present disclosure. In addition to the headphones 110 and wearable device 210, system 250 also comprises a processing host 230 incorporating a pre-processing module 230A and a processing module 230B.

Like system 150, system 250 utilizes the headphones 110 that isolate the right and left heartbeat signals, the PCG extraction module 120 generates the right and left PCG signals PCG1, PCG1 from the isolated heartbeat signals, and a processing host device 230 that generates the blood pressure, heart-related biometric data, and/or heart condition diagnoses. For the sake of brevity, the details of the PCG1, PCG2 signal processing generated by headphones 110 and PCG extraction module 120 will not be repeated, as such details have been comprehensively disclosed above in the description of system 150.

As shown, the wearable device 210 of system 250 generates a third heart-beat related signal 215 that is to be supplied to processing host 230. As noted above, wearable device 210 includes sensor(s) configured to detect and generate the third heart-related signal 215 for processing. The third heart-related signal 215 may comprise a PCG, which as noted above, is a signal that records heart sounds during the cardiac cycle, an Electrocardiography (ECG) that records electrical activity of the heart, or Photoplethysmography (PPG) that records volumetric blood changes during circulation.

The pre-processing module 230A is configured to perform various signal processing steps on the received independent right and left PCG1, PCG2 signals as well as the received third signal 215 from wearable device 210. As noted above, such signal processing steps may include filtering, amplification, noise mitigation, etc. to determine the two PTT11, PTT12 signals from two different body location points.

In addition, the pre-processing module 230A also processes the third signal 215 from wearable device 210 to generate PTT2, PTT3 signals from the wearable device 210. That is, because PTT signals require two different body location points, the third signal 215 is process by associating it with the right side headphone speaker to generate a PTT2 signal as well as being processed by associating it with the left side headphone speaker to generate a PTT3 signal.

Furthermore, as noted above regarding pre-processing module 130A, pre-processing module 230A may also incorporate MD techniques to identify any heart murmurs and/or heart valve issues, based on the PCG 1, PCG 2, and third 215 signals. Accordingly, pre-processing module 230A operates to generate PTT11, PTT12, PTT2, PTT3, and MD signals.

In the depicted embodiment, the MD signal is supplied to processing module 230B while the PTT11, PTT12, PTT2, PTT3 signals are supplied to PTT-to-PWV converter module 230C. That is, the PTT11, PTT12, PTT2, PTT3 signals are converted to Pulse Wave Velocity (PWV) signals that are directly related to the blood pressure. The converted PTT11, PTT12, PTT2, PTT3 signals are then supplied to processing module 230B. In some embodiments, additional biometric data, such as, diastolic DP1, DP2 period data, systolic SP1, SP2 period data, and PCG1, PCG2 signals may also be supplied to the processing module 230B.

Processing module 230B configured to provide additional signal processing steps, such as, filtering, amplification, noise mitigation, timing correlations, etc. as well as applying AI deep learning generated algorithms to identify the heart-related biometric data that may indicate any heart-related conditions, such as, blood pressure measurements along with heart murmur, heart valve, and blood vessel issue detection. Accordingly, FIG. 2B illustrates an exemplary graph 240 representing the heart-related data, in waveform fashion, generated by processing module 230B.

As shown, graph 240 depicts data representing the PCG1, PCG2, PCG, PPG, ECG data. In addition to the PCG1, PCG2, PTT11, PTT12, systolic period data SP1, SP2, and diastolic period data DP1, DP2, as noted above regarding the data provided by graph 140 of FIG. 1C, graph 240 further provides data regarding determination of PTT2, PTT3 signals based on the PCG, PPG, ECG data.

In this manner, system 250 provides the heart-related biometric data, namely, PTT11, PTT12, PTT2, PTT3, MD, SP1, SP2, DP1 and DP2 signals to enable the diagnoses of heart-related conditions without subjecting patients to intrusive or movement-restrictive procedures. Such heart-related conditions may include, but are not limited to, blood pressure measurements along with heart murmur, heart valve, and blood vessel issue detection.

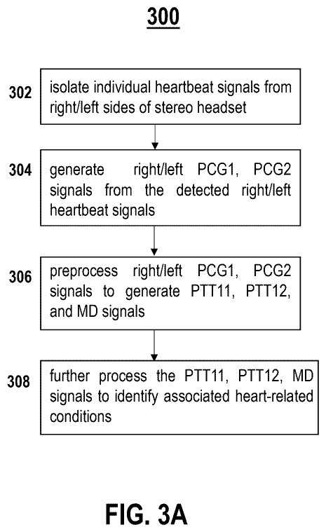

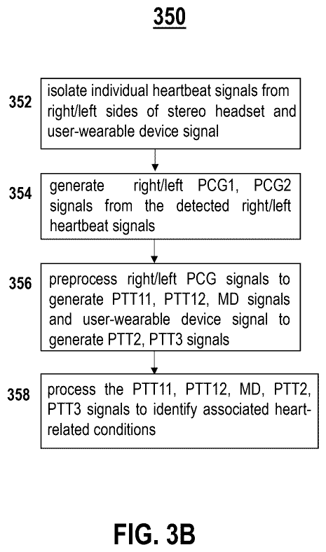

FIG. 3A depicts a flowchart of a method 300 for determining blood pressure and heart-related biometric data based on heartbeat signals detected by an earpiece device (e.g., headphones) 110, in accordance with the embodiments of the present disclosure.

Method 300 commences at task block 302, in which the headphones 110 isolates right and left heartbeat signals. At task block 304, right/left PCG1, PCG2 signals are generated from detected right/left heartbeat signals. As detailed above, the PCG extraction module 120 is configured to generate the right and left PCG1, PCG2 signals based on the detected right/left heartbeat signals.

At task block 306, the PCG1, PCG2 signals are preprocessed to generate PTT11, PTT12, and MD signals. As detailed above, pre-processing module 130A may apply filtering, amplification, and noise mitigation signal processing techniques as well as MD techniques to generate the PTT11, PTT12, and MD signals.

At task block 308, the PTT11, PTT12, and MD signals are further processed to identify any heart-related conditions. As detailed above, processing module 130B may apply filtering, amplification, and noise mitigation signal processing techniques as well as AI deep learning generated algorithms configured to identify any heart-related conditions associated with the PTT11, PTT12, and MD signals, such as, blood pressure measurements along with heart murmur, heart valve, and blood vessel issue detection.

FIG. 3B depicts a flowchart of a method 350 for determining blood pressure and heart-related biometric data based on heartbeat signals detected by the earpiece device (e.g., headphones) 110 as well as the user-wearable device 210, in accordance with the embodiments of the present disclosure.

Method 350 commences at task block 352, in which the headphones 110 isolates right and left heartbeat signals and a user-wearable device 210 that provides a third heart-related signal 215. As noted above, the third heart-related signal 215 may comprise a PCG signal, an ECG signal or a PPG signal.

At task block 354, right/left PCG1, PCG2 signals are generated from detected right/left heartbeat signals. As detailed above, the PCG extraction module 120 is configured to generate the right and left PCG1, PCG2 signals based on the detected right/left heartbeat signals.

At task block 356, the PCG1, PCG2 signals are pre-processed to generate PTT11, PTT12, and MD signals while third heart-related signal 215 is pre-processed to generate PTT2, PTT3 signals. As noted above, the third signal 215 is associated with the right side headphone speaker to generate a PTT2 signal and is associated with the left side headphone speaker to generate a PTT3 signal. The pre-processing module 230A may apply filtering, amplification, and noise mitigation signal processing techniques as well as MD techniques to generate the PTT11, PTT12, MD, PTT2, PTT3 signals.

At task block 358, the PTT11, PTT12, PTT2, PTT3, and MD signals are further processed to identify any heart-related conditions. As detailed above, processing module 230B may apply filtering, amplification, and noise mitigation signal processing techniques as well as AI deep learning generated algorithms configured to identify any heart-related conditions associated with the PTT11, PTT12, PTT2, PTT3, and MD signals, such as, blood pressure measurements along with heart murmur, heart valve, and blood vessel issue detection.

It will be appreciated that, while the disclosed embodiments have been described in terms of system configurations/components for clarity and tractability, the related methods and processes regarding the execution of the operations of the disclosed configurations/components should be clearly understood by artisans of ordinary skill in the art.

With this said, modifications and improvements to the above-described implementations of the present technology may become apparent to those skilled in the art. The foregoing description is intended to be exemplary rather than limiting. The scope of the present technology is therefore intended to be limited solely by the scope of the appended claims.

### BIBLIOGRAPHY

[1] Mohammed Nabih-Ali, El-Sayed A. El-Dahshan, and Ashraf S. Yahia. Heart diseases diagnosis using intelligent algorithm based on pcg signal analysis. Circuits and Systems, 08:184-190, 2017. 1, 5;

[2] Piyush Sharma, Syed Anas Imtiaz, and Esther Rodriguez-Villegas. Acoustic sensing as a novel wearable approach for cardiac monitoring at the wrist. Scientific Reports, 9, 12 2019. 1, 5;

[3] Josep Solà and Ricard Delgado-Gonzalo. The Handbook of Cuffless Blood Pressure Monitoring A Practical Guide for Clinicians, Researchers, and Engineers. 1 2019. 1, 2;

[4] Ramakrishna Mukkamala, Jin Oh Hahn, Omer T. Inan, Lalit K. Mestha, Chang Sei Kim, Hakan Toreyin, and Survi Kyal. Toward ubiquitous blood pressure monitoring via pulse transit time: Theory and practice. IEEE Transactions on Biomedical Engineering, 62:1879-1901, 8 2015. 1;

[5] Carmel M. McEniery, John R. Cockcroft, Mary J. Roman, Stanley S. Franklin, and Ian B. Wilkinson. Central blood pressure: Current evidence and clinical importance, 7 2014; and

[6] International Patent Application Publication No. WO 2021/237206 A1 to FAN Xiaoran et al. (hereinafter “FAN”), published on Nov. 25, 2021.

All of the identified references [1], [2], [3], [4] and [5] are incorporated by reference herein in their entireties.

What is claimed is:

1. A system for determining heart-related biometric data, comprising:
an earpiece device configured to receive audio signals and isolate right and left heartbeat signals from the received audio signals;
a first processor communicatively-coupled to the earpiece device and configured to process the isolated right and left heartbeat signals to generate respective right and left phonocardiogram PCG1, PCG2 data signals; and
a processing host comprising a second processor configured to:
perform signal processing measures on the right and left PCG1, PCG2 signals to determine respective pulse travel time PTT11, PTT12 data signals, and
perform signal processing measures on the pulse travel time PTT11, PTT12 data signals to provide an indication of heart-related conditions.

2. The system of claim 1, wherein the earpiece device comprises headsets, headphones, hearing aids, earbud set that is configured to provide independent right and left audio signals and isolate right and left heartbeat signals from the received audio signals.

3. The system of claim 1, wherein the processing host comprises a computer, a smartphone, or a smart watch.

4. The system of claim 1, wherein the second processor is further configured to perform signal processing measures that include morphological detection (MD) techniques on the PCG1, PCG2, PTT11, PTT12 data signals to generate an MD data signal indicative of heart murmur and/or related heart valve issues.

5. The system of claim 1, wherein the second processor further generates diastolic period DP1, DP2 data signals and systolic period SP1, SP2 data signals, based on the PCG1, PCG2, PTT11, PTT12 data signals.

6. The system of claim 1, wherein the signal processing measures of the second processor further incorporate artificial intelligence (AI) deep-learning generated algorithms to the PTT11, PTT12 data signals, and/or MD, DP1, DP2, SP1, SP2 data signals to provide an indication of heart-related conditions.

7. The system of claim 1, further comprising a user-wearable device including one ore more sensors configured to detect and generate a third heart-related signal for processing by the second processor.

8. The system of claim 7, wherein third heart-related signal comprises a phonocardiogram PCG data signal, an electrocardiography ECG data signal, or a photoplethysmography PPG data signal.

9. The system of claim 7, wherein the second processor processes the third heart-related signal with the right-side speaker of the earpiece device to generate a PTT2 data signal and processes the third heart-related signal with the left-side speaker of the earpiece device to generate a PTT3 data signal.

10. The system of claim 7, wherein the second processor is further configured to convert the PTT11, PTT12, PTT2, PTT3 data signals into PWV data signals indicative of blood pressure values.

11. The system of claim 10, wherein the PWV data signals and the MD data signal are supplied to the second processor to determine heart-related conditions.

12. A method for determining heart-related biometric data, comprising:
receiving, from an earpiece device, audio signals;
isolating right and left heartbeat signals from the received audio signals;
generating right and left phonocardiogram signals PCG1, PCG2 from the isolated right and left heartbeat signals, respectively;
pre-processing, the right and left PCG1, PCG2 signals to determine respective pulse travel time signals PTT11, PTT12 data signals; and
processing, the PTT11, PTT12 data signals to provide an indication of heart-related conditions.

13. The method of claim 12 further comprising applying morphological detection (MD) to the PCG1, PCG2, PTT11, PTT12 data signals to generate an MD data signal indicative of heart murmurs.

14. The method of claim 12, further comprising generating diastolic period DP1, DP2 data signals and systolic period SP1, SP2 data signals based on the PCG1, PCG2, PTT11, PTT12 data signals.

15. The method of claim 12, further comprising applying artificial intelligence (AI) deep-learning generated algorithms to the PTT11, PTT12 data signals, and/or MD, DP1, DP2, SP1, SP2 data signals to provide an indication of heart-related conditions.

16. The method of claim 12, further comprising:
generating, by a user-wearable device containing sensor(s), a third heart-related signal; and
performing pre-processing and processing of the third heart-related signal.

17. The method of claim 16, wherein the pre-processing of the third heart-related signal includes processing the third heart-related signal with the right-side speaker of the earpiece device to generate a PTT2 data signal and processing the third heart-related signal with the left-side speaker of the earpiece device to generate a PTT3 data signal.

18. The method of claim 16, further comprising converting, the PTT11, PTT12, PTT2, PTT3 data signals into PWV data signals indicative of blood pressure values.

19. The method of claim 16, further comprising processing the PWV data signals and the MD data signal to determine heart-related conditions.

20. A non-transitory computer-readable medium comprising executable instructions which, when executed by at least one processor, cause the at least one processor to carry out steps of a method for determining heart-related biometric data, the method comprising:
receiving, from an earpiece device, audio signals;
isolating right and left heartbeat signals from the received audio signals;
generating right and left phonocardiogram signals PCG1, PCG2 from the isolated right and left heartbeat signals, respectively;
pre-processing, the right and left PCG1, PCG2 signals to determine respective pulse travel time signals PTT11, PTT12 data signals; and
processing, the PTT11, PTT12 data signals to provide an indication of heart-related conditions.
